(19) United States
(12) Patent Application Publication
(10) Pub. No.: US 2010/0016378 A1
(43) Pub. Date: Jan. 21, 2010

(54) Method of preventing dihydropyridine compound from degradation

(76) Inventor: Toshio Suzuki, Saitama (JP)

Correspondence Address: BIRCH STEWART KOLASCH & BIRCH, PO BOX 747, FALLS CHURCH, VA, 22040-0747, US

(21) Appl. No.: 11/919,232

(22) PCT Filed: Apr. 27, 2006
(86) PCT No.: PCT/JP2006/308869

§ 371 Date: Oct. 25, 2007

(30) Foreign Application Priority Data
Apr. 28, 2005 (JP) 2005-131906

(51) Int. Cl. A61K 31/4418

(52) U.S. Cl. 514/356

(57) ABSTRACT

As the addition of water to dihydropyridine compounds such as amlodipine accelerates their degradation into the pyridine form, either immediately or following the formation of unstable hydrates, methods for producing pharmaceutical compositions that contain dihydropyridine compounds are limited to methods in which no water is added, such as methods in which dihydropyridine compounds are added in the form of powder, but these methods are unsuitable for wet granulation in which tablet moldability, hardness, disintegration, or the like can be readily adjusted and the homogeneity of the ingredients can be readily ensured by modifying the granulating conditions. According to the present invention, the co-presence of a methylated cellulose-based polymer during wet processing of dihydropyridine compounds prevents the formation of dihydropyridine compound hydrates and/or the degradation of the dihydropyridine compounds.

### TECHNICAL FIELD

The present invention relates to a method for stabilizing dihydropyridine compounds, and more particularly to a method for preventing formation of hydrates of dihydropyridine compounds when dihydropyridine compounds are wet processed, as well as to pharmaceutical compositions and the like that are obtained by wet processing dihydropyridine compounds.

### BACKGROUND ART

The populations of the major industrialized nations, especially Japan, are rapidly aging, and more than half of patients with hypertension are said to be elderly individuals aged 70 years or more. As an excessive decrease in blood pressure resulting from the administration of antihypertensive medication is particularly undesirable for elderly individuals, the drug of first choice for hypertension in elderly patients is now dihydropyridine calcium antagonists, which have a more moderate onset of efficacy and longer-lasting efficacy. At present, amlodipine besylate, manidipine hydrochloride, nicardipine hydrochloride, and the like are commercially available as dihydropyridine compounds with calcium antagonism. Of these, amlodipine besylate is the leading compound in Japan, and is commercially available in the form of film-coated tablets.

Tablets are a frequently used dosage form for pharmaceutical products because they are convenient to handle and easy to take, etc. Recently, in particular, rapidly disintegrating tablets that readily disintegrate in the mouth cavity, which have been developed as a dosage form that is easier for pediatric patients and elderly patients who have difficulty swallowing to take without water, have become more important. Examples of rapidly disintegrating tablets containing a dihydropyridine compound include those that are obtained by a process comprising granulating mannitol, crystalline cellulose, the disintegrant sodium carboxymethyl starch, or the like, followed by adding and mixing with powder such as amlodipine besylate, the disintegrant croscarmellose sodium, and the like, and direct tableting (Patent Document 1).

Regarding the stability of amlodipine besylate, it is reported that lactose, which is commonly used as an excipient, degrades amlodipine besylate and should therefore be avoided (Non-Patent Document 1). As a way of coping with this problem, Patent Document 2 discloses tablets that are obtained by direct tableting, containing crystalline cellulose blended in a high concentration of 87 to 94% in a pharmaceutical composition.

In both Patent Documents 1 and 2, tablets containing a dihydropyridine compound are obtained by direct tableting. Although it is advantageous in that the process is simple, direct tableting has a problem that it is difficult to achieve a homogenous mixture when using dihydropyridine compound such as amlodipine besylate which do not have very good fluidity, as the ingredients are mixed in the form of powder.

In the meantime in wet granulation, it is easy to adjust tablet moldability, hardness, disintegration, and the like, by modifying the granulating conditions and it is also easy to ensure the homogeneity of the ingredients. It is desirable to employ wet granulation particularly in preparing high quality tablets such as rapidly disintegrating tablets. Particularly because pharmaceutical compositions containing a dihydropyridine compound such as amlodipine besylate are frequently taken by elderly patients with hypertension, it would be preferable to design dosage forms which are easy to administer even to patients who have difficulty swallowing, or dosage forms which permit the dosage to be readily adjusted, thus making them suitable for cautious administration to the elderly.

Patent Document 1: Japanese Unexamined Patent Application (Kokai) H10-298062

Patent Document 2: WO 03/051364

Non-Patent Document 1: M. Abdoh et al., “Amlodipine Besylate: Excipients Interaction in Solid Dosage Form,” Pharmaceutical Development and Technology, (USA), 2004, Vol. 9, No. 1, pp. 15-24

### DISCLOSURE OF INVENTION

### Problem to be Solved by the Invention

However, as addition of water accelerates the degradation of dihydropyridine compounds into the pyridine form, either immediately or following the formation of unstable hydrates, methods for preparing pharmaceutical compositions containing dihydropyridine compounds are limited to methods in which no water is added, such as methods in which dihydropyridine compounds are added in the form of powder, as described, for example, in Patent Documents 1 and 2. It has thus been difficult to obtain high quality tablets such as rapidly disintegrating tablets. An object of the present invention is to provide a method for ensuring the stability of dihydropyridine compounds in pharmaceutical compositions obtained by a process in which water is added (wet processing), such as wet kneading, wet granulation, or fluid bed granulation; pharmaceutical compositions with improved dihydropyridine compound stability obtained by such a method; and dihydropyridine compound-containing pharmaceutical compositions that are easy to take and convenient to handle.

### Means for Solving the Abovementioned Problems

As a result of extensive research to address the above-identified problem, the inventors of the present invention found that the stability of dihydropyridine compounds in pharmaceutical compositions obtained by wet processing could be vastly improved by wet processing the dihydropyridine compounds with a methylated cellulose-based polymer. The inventors also discovered that the above method prevents formation of dihydropyridine compound hydrate crystals and amorphous types as well as subsequent degradation to pyridine compounds. That is, the present invention relates to a method of stabilizing a dihydropyridine compound, comprising wet processing the dihydropyridine compound with a methylated cellulose-based polymer; a method of preventing formation of hydrate crystals from a wet processed dihydropyridine compound and/or degradation of a dihydropyridine compound, comprising wet processing the dihydropyridine compound with a methylated cellulose-based polymer; a pharmaceutical composition with improved dihydropyridine compound stability, obtained by wet processing dihydropyridine compound with the use of a methylated cellulose-based polymer; and the like.

### EFFECT OF THE INVENTION

The present invention can prevent formation of unstable hydrates in pharmaceutical compositions containing dihydropyridine compounds, thereby enabling the compositions to be kneaded with the use of water and also enabling the composition to be granulated by wet granulation, which is a common process, other than the process that does not involve the use of water such as direct tableting and dry granulation. It is also possible to provide pharmaceutical compositions in which the stability of dihydropyridine compounds can be ensured by using the methods.

### BEST MODE FOR CARRYING OUT THE INVENTION

The present invention is described below on the basis of preferred embodiments.

Dihydropyridine Compounds and Amlodipine

The dihydropyridine compounds used in the present invention are compounds having a dihydropyridine skeleton, such as amlodipine, azelnidipine, barnidipine, benedipine, efonidipine, manidipine, and nicardipine. A preferred dihydropyridine is amlodipine.

Amlodipine

In the present invention, amlodipine can be used in free form as well as in the form of various salts. As used in the present Description, the term “amlodipine” means both of that in the free form and that in the form of any salt, unless otherwise expressly specifying the salt. Amlodipine besylate, the compound represented by the following formula, is the preferred amlodipine. Amlodipine besylate produced by Dr. Reddy's Company is commercially available.

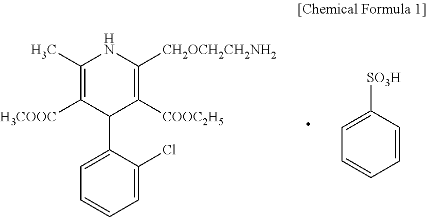

Methylated Cellulose-Based Polymer

Examples of the methylated cellulose-based polymer used in the present invention include methyl cellulose and hydroxypropylmethyl cellulose.

Methyl Cellulose and Hydroxypropylmethyl Cellulose

The methyl cellulose which can be used in the present invention can be classified into various grades depending on its viscosity. Any grade can be used in the present invention. Examples of commercially available methyl cellulose which can be used in the present invention include Metolose (produced by Shin-Etsu Chemical Co., Ltd.), Methocel A (produced by The Dow Chemical Company), and Marpolose (produced by Matsumoto Yushi-Seiyaku Co., Ltd.). The hydroxypropylmethyl cellulose which can be used in the present invention can be classified into various grades depending on its viscosity, the degree of methyl substitution, and the degree of hydroxypropyl substitution. Any grade can be used in the present invention. Examples of commercially available hydroxypropylmethyl cellulose which can be used in the present invention include Metolose 65SH (produced by Shin-Etsu Chemical Co., Ltd.) and Methocel K (produced by The Dow Chemical Company). Methods for adding the methylated cellulose-based polymer during wet is kneading or wet granulation in the present invention include, but are not limited to, a method comprising dissolving or suspending the methylated cellulose polymer in aqueous solution, followed by adding the solution or suspension, or adding the methylated cellulose polymer in the form of powder. The methylated cellulose polymer is preferably added by dissolving in water.

Amount of Methyl Cellulose Added

The amount of methylated cellulose polymer used in the present invention will vary depending on the process that is used, the dosage form, and the like, and is usually, for example, at least 0.000005 weight part per one weight part dihydropyridine compound (based on weight including salt, if a salt is formed). At least 0.00005 weight part is preferable, and at least 0.0005 weight part is even more preferable. Commonly used additives such as binders, disintegrants, stabilizers, excipients, plasticizers, fragrances, and sweeteners other than the dihydropyridine compound and methylated cellulose polymer can also be optionally used in the present invention.

The wet processing used in the present invention means that the processing where the dihydropyridine compound is processed in hydrous form and is not limited to a particular process. Examples of wet processing include wet kneading, where a powder containing the dihydropyridine compound is kneaded with the addition of a solution containing the methylated cellulose compound; wet granulation, where the mixture is kneaded, and wet granules are then produced; and fluid bed granulation, where a dihydropyridine compound-containing powder fluidized by hot air is sprayed with a solution containing the methylated cellulose compound. Wet granulation can be more specifically classified into methods such as mixed agitation granulation employing a planetary mixer, screw mixer, or the like; high speed mixed agitation granulation employing a Henschel mixer, super mixer, or the like; extrusion granulation employing a cylindrical granulator, rotary granulator, screw extruder granulator, pellet mill granulator; rotary granulation; fluid bed granulation; or spray granulation Any method can be employed in the present invention.

The water used for wet processing in the present invention includes, but is not limited to, purified water commonly used in the manufacture of pharmaceutical products and the like. When water is added for wet processing, the water can be added alone or it can be added in the form of a solvent mixture comprising an organic solvent such as an alcohol mixed with water. In such cases, the water (solvent containing water) may be added alone, or it may be added in the form of a solution of soluble elements or a suspension of insoluble elements.

Pharmaceutical Compositions

The form of the pharmaceutical composition of the invention is not limited, provided that it is a pharmaceutical composition which has been obtained by wet processing. Examples include, but are not limited to:

(1) granulated materials obtained by wet granulation or fluid bed granulation;

(2) dry syrups obtained by lyophilization of aqueous solutions or suspensions comprising a dihydropyridine compound and methylated cellulose polymer;

(3) granules or fine particles obtained by drying the granulated materials of (1);

(4) tablets or oral disintegrating tablets obtained by tableting the granulated material of (1) immediately or after the addition of other additives;

(5) tablets or oral disintegrating tablets obtained by tableting the granules or fine particles of (3) immediately or after the addition of other additives; and

(6) capsules filled with the granules or fine particles of (3).

### EXAMPLES

The following examples further illustrate the present invention, but are not intended to limit the scope thereof.

### Example 1

60 g of mannitol (produced by Towa Chemical Industry Co., Ltd.) was added to 3.5 g of amlodipine besylate (produced by Dr. Reddy's Company), and mixed in a Mechanomill (produced by Okada Seiko Co., Ltd.). The mixture was wet kneaded using 8.95 g of a 2.9% methyl cellulose (Metolose 25, produced by Shin-Etsu Chemical Co., Ltd.) aqueous solution. The kneading was followed by fluid bed drying at an air intake temperature of 90° C. with a Flow Coater (produced by Freund/Okawara). The product was sifted on a sieve to give granules.

### Example 2

### Agitation Granulation

20 g of crystalline cellulose (Avicel PH301, by Asahi Kasei Co., Ltd.) was added to 20 g of amlodipine besylate, and kneaded in a Mechanomill. The mixture was wet kneaded using 18.2 g of a 2.9% methyl cellulose aqueous solution. The kneading was followed by fluid bed drying at an air intake temperature of 90° C. with a Flow Coater. The product was sifted on a sieve to give granules.

### Example 3

### Fluid Bed Granulation

250 g of crystalline cellulose was added to 250 g of amlodipine besylate, and mixed in a plastic bag. Granules were obtained by fluid bed granulation at an air intake temperature of 90° C. with a Flow Coater using 352 g of 2.9% methyl cellulose aqueous solution.

### Comparative Example 1

60 g of mannitol was added to 3.5 g of amlodipine besylate, and mixed in a Mechanomill. The mixture was then wet kneaded using 8 g of purified water. The kneading was followed by fluid bed drying at an air intake temperature of 90° C. with a Flow Coater. The product was sifted on a sieve to give granules.

### Examples 4 through 9, Comparative Examples 3 and 4

### Rapidly Disintegrating Tablet

Amlodipine besylate, mannitol, and in some cases yellow iron sesquioxide (by Junsei Chemical Co., Ltd.), were mixed in the proportions given in Tables 1 and 2 in a Mechanomill, and the mixtures were kneaded in a methyl cellulose aqueous solution or an ethanol aqueous solution comprising polyvinyl pyrrolidone (by ISP Co., Ltd., hereinafter referred to as “PVP”) or polyvinyl alcohol (by Nippon Fats & Oils Co., Ltd., hereinafter referred to as “PVA”). The resulting moist powder was tableted using an EMP rapidly disintegrating tableting system (device disclosed in Japanese Patent No. 3,179,658) and dried to give rapidly disintegrating tablets. The diameter of the tablet was 8 mm to 8.5 mm, the thickness of the tablet was 3.2 mm to 3.4 mm, the molding pressure was 15 kgf, and the drying temperature was 90° C.

TABLE 1

| | Example 4 | Example 5 | Example 6 | Example 7 | Example 8 | Example 9 |
|---|---|---|---|---|---|---|
| Amlodipine besylate | 10.4 g | 10.4 g | 10.4 g | 10.4 g | 10.4 g | 10.4 g |
| Mannitol | 281.1 g | 281.1 g | 281.1 g | 281.1 g | 281.1 g | 281.1 g |
| methyl cellulose | 0.072 g | 0.36 g | 0.72 g | 0.96 g | 1.2 g | 1.44 g |
| yellow iron sesquioxide | 8.5 g | 8.5 g | 8.5 g | 8.5 g | 8.5 g | 8.5 g |

TABLE 2

| | Comparative Ex. 3 | Comparative Ex. 4 |
|---|---|---|
| Amlodipine besylate | 16.3 g | 16.3 g |
| Mannitol | 775.7 g | 778.9 g |
| PVP | 8.0 g | — |
| PVA | — | 4.8 g |

### Examples 10 and 11, and Comparative Examples 5 Through 7

(1) 5 g of amlodipine besylate and (2) 5 g of the methyl cellulose, hydroxypropylmethyl cellulose (TC-5RW, by Shin-Etsu Chemical), PVP, hydroxypropyl cellulose (HPC-L, by Nippon Soda Co., Ltd.), or polyethylene glycol (PEG6000, by Nippon Fats & Oils Co., Ltd.) given as additives in Table 3 below were mixed to homogeneity with a mortar. 25% ethanol aqueous solution was added, and kneaded to give a kneaded product. The kneaded product was dried for 10 hours at 40° C. in a bench-top dryer (DAE-20 model hot air dryer, by Sanwa Kaki Kogyo Co., Ltd.) to give granules.

TABLE 3

| | Additives |
|---|---|
| Example 10 | methyl cellulose |
| Example 11 | hydroxypropylmethyl cellulose |
| Comparative Example 5 | polyvinyl pyrrolidone |
| Comparative Example 6 | hydroxypropyl cellulose |
| Comparative Example 7 | polyethylene glycol |

The granules obtained in Examples 1, 2, and 3 and Avicel PH101 or Avicel PH101/Mannitol P (weight ratio 4:6 mixture) were measured into plastic bags in the proportions given in Table 4 and were thoroughly mixed in the plastic bags, and the mixtures were molded at a tableting pressure of 300 kgf using a Shimadzu Autograph AGS-1000D to give approximately 180 mg tablets containing the active ingredient in an amount of 5 mg or 2.5 mg. The material was tableted using an 8 mm mortar and pestle coated with magnesium stearate.

TABLE 4

| | | | Content of |
|---|---|---|---|
| | | | amlodipine |
| | | | besylate active |
| | | | ingredient |
| No. | Granules | Excipient | per tablet |
| 1 | Example 1 granules | Avicel PH101 | 5 mg |
| | 1.26 g | 0.54 g | |
| 2 | Example 2 granules | Avicel PH101/MannitolP | 5 mg |
| | 139 mg | 1.66 g | |
| 3 | Example 3 granules | Avicel PH101/MannitolP | 2.5 mg |
| | 70.8 mg | 1.729 g | |

### Examples 12, 13, 14, and Comparative Example 8

0.1 g of water or methylated cellulose polymer aqueous solution (MC aqueous solution) of the type and concentration given in Tables 5 through 8 below was added to 0.5 g of amlodipine besylate, and kneaded in a mortar. After being kneaded, the material was dried at room temperature to give granules.

TABLE 5
Control

| | MC aqueous solution | Amount of MC added (weight |
|---|---|---|
| | concentration (wt %) | part/1 weight part AML) |
| Comparative | 0 (water) | 0 |
| Ex. 8 | | |

TABLE 6
Methyl cellulose (Shin-Etsu Chemical, Metolose SM-25)

| | MC aqueous solution | Amount of MC added (weight |
|---|---|---|
| | concentration (wt %) | part/1 weight part AML) |
| Example 12-1 | 2.9 | 5.8 × 10⁻³ |
| Example 12-2 | 0.29 | 5.8 × 10⁻⁴ |

TABLE 7
Methyl cellulose (Shin-Etsu Chemical, Metolose SM-4)

| | MC aqueous solution | Amount of MC added (weight |
|---|---|---|
| | concentration (wt %) | part/1 weight part AML) |
| Example 13-1 | 2.9 | 5.8 × 10⁻³ |
| Example 13-2 | 0.29 | 5.8 × 10⁻⁴ |
| Example 13-3 | 0.029 | 5.8 × 10⁻⁵ |

TABLE 8
Hydroxypropylmethyl cellulose (Shin-Etsu
Chemical, Metolose 60SH-50)

| | MC aqueous solution | Amount of MC added (weight |
|---|---|---|
| | concentration (wt %) | part/1 weight part AML) |
| Example 14-1 | 2.9 | 5.8 × 10⁻³ |
| Example 14-2 | 0.29 | 5.8 × 10⁻⁴ |

(Test)

The stability of amlodipine besylate in the granules, tablets, and the like obtained in the preceding examples and comparative examples were tested (accelerated tests). The following procedures were used to determine the purity and crystal form under extreme conditions and in the following tests.

(Extreme Test Conditions)

Transparent glass vials (sealed or open) or Petri dishes were used as the storage containers. Samples were uniformly spread in the Petri dishes. For the stability tests, thermo-hygrostatic chambers were used under the following conditions. 40° C. and 75% relative humidity (RH): FX230p (by ETAC Company); 60° C.: DN94 (by Yamato Co., Ltd.).

(Method for Determining Purity)

The impurity content of pharmaceutical compositions containing amlodipine besylate was assessed by the gradient concentration regulations (gradient method) of the liquid chromatography methods given in the Japanese Pharmacopoeia. The area percent method (relative area method) was used to calculate the numerical values. The liquid chromatograph (HPLC) of Hitachi Ltd. D-7000 was used under the following conditions.

<HPLC Conditions>

Detector: UV spectrophotometer (measuring wavelength: 241 nm); column: Inertsil ODS2 4.6 mm×15 cm; particle diameter: 5 μm; column temperature: 40° C.; flow rate: 0.9 mL/min; injection volume: 10 μL; analysis time: 45 min; HPLC mobile phase A: acetonitrile/water/HClO₄ (100:900:1); HPLC mobile phase B: acetonitrile/water/HClO₄ (900:100:1); gradient program: as shown in Table 9.

TABLE 9

| | Mobile phase B |
|---|---|
| Time | concentration |
| (min) | (%) |
| 0 | 30 |
| 15 | 30 |
| 35 | 100 |
| 35.01 | 30 |
| 45 | Stop |

<Preparation of Test Solution>

The sample was measured out and dissolved with the addition of 30% acetonitrile solution, and the concentration of the solution was adjusted to 0.7 mg/mL based on the mass of bulk drug. Sample solution was prepared by centrifugation (3000 rpm, 10 min) or filtration (4 mL of initial fraction was eliminated) as needed.

(Measurement of Crystal Form)

The crystal form of amlodipine besylate contained in the pharmaceutical compositions was assessed by differential scanning calorimetry (DSC) method which is the thermal analysis method given in the Japanese Pharmacopoeia. Measurements were taken with a DSC3100S (by MAC Science Company) from room temperature to 210° C. at a heating rate of 5° C./min or 1° C./min. Samples were measured out into aluminum containers which were then sealed. The control standard substance was an empty aluminum container.

(Assessment of Crystal Form)

The following findings had been obtained under the above conditions, and the crystal form was determined on the basis of these findings.

1. Anhydrous amlodipine besylate crystals do not exhibit crystal transition until decomposition point around 200° C.

2. Monohydrate crystals and dihydrate crystals exhibited endothermic peaks associated with the separation of water between room temperature and 100° C., and endothermic peaks associated with crystal transition to anhydrous crystals at 120° C. or above.

(Test 1)

Storage stability tests were conducted for one month using the granules obtained in Examples 1 through 3 and Comparative Example 1 in a 40° C. 75% RH open system and a 60° C. closed system, and the impurity content and the crystal form of the amlodipine besylate in the granules were determined. The results of the storage stability tests are given in Table 10.

TABLE 10

| | | Amount of Degradation |
|---|---|---|
| | Storage conditions | product after 1 month |
| Example 1 | 40° C. 75% RH, open | <0.05% |
| | 60° C., closed | 0.05% |
| Example 2 | 40° C. 75% RH, open | <0.05% |
| | 60° C., closed | <0.05% |
| Example 3 | 40° C. 75% RH, open | <0.05% |
| | 60° C., closed | <0.05% |
| Comparative Ex. 1 | 40° C. 75% RH, open | 0.15% |
| | 60° C., closed | 10.21% |

The granules obtained in Examples 1 through 3 were found to be more stable under all conditions than the granules obtained in Comparative Example 1. The crystal form of the granules obtained in Example 1 and Comparative Example 1 was also measured. The amlodipine besylate was in the form of anhydrous crystals in Example 1, which was granulated using methyl cellulose, and the amlodipine besylate was amorphous in Comparative Example 1, which was granulated without using methyl cellulose. The results of these tests show that granules having stable anhydrous amlodipine besylate crystals that are not affected by the temperature and humidity of the storage environment can be provided by kneading, granulating and drying amlodipine besylate with the use of a methyl cellulose.

(Test 2)

Stability tests were conducted, in the same manner as in Test Example 1, on the tablets obtained in Examples 4 through 9 and Comparative Examples 3 and 4. The results are given in Table 11.

TABLE 11

| | | Amount of Degradation |
|---|---|---|
| | Storage conditions | product after 1 month |
| Example 4 | 40° C. 75% RH, open | <0.05% |
| | 60° C., closed | <0.05% |
| Example 5 | 40° C. 75% RH, open | <0.05% |
| | 60° C., closed | <0.05% |
| Example 6 | 40° C. 75% RH, open | <0.05% |
| | 60° C., closed | <0.05% |
| Example 7 | 40° C. 75% RH, open | <0.05% |
| | 60° C., closed | <0.05% |
| Example 8 | 40° C. 75% RH, open | <0.05% |
| | 60° C., closed | <0.05% |
| Example 9 | 40° C. 75% RH, open | <0.05% |
| | 60° C., closed | <0.05% |
| Comparative Ex. 3 | 40° C. 75% RH, open | 0.47% |
| | 60° C., open | 4.38% |
| Comparative Ex. 4 | 40° C. 75% RH, open | 0.06% |
| | 60° C., open | 12.88% |

The crystal form of the amlodipine contained in the tablets obtained in Examples 4 through 9 and Comparative Example 3 were also measured. The results show that the amlodipine besylate in the tablets obtained with the use of methyl cellulose during wet kneading (Examples 4 to 9) were in the form of anhydrous crystals, and that the amlodipine besylate in the tablets obtained with the use of PVP during wet kneading (Comparative Example 3) was in the form of hydrate crystals.

(Test 3)

Stability tests were conducted on the granules obtained in Examples 10 and 11 and Comparative Examples 5 to 7. The results are given in Table 12.

TABLE 12

| | | Amount of Degradation |
|---|---|---|
| | Storage conditions | product after 1 month |
| Example 10 | 40° C. 75% RH, open | <0.05% |
| | 60° C., closed | <0.05% |
| Example 11 | 40° C. 75% RH, open | <0.05% |
| | 60° C., closed | <0.05% |
| Comparative. Ex. 5 | 40° C. 75% RH, open | 0.06% |
| | 60° C., closed | 0.25% |
| Comparative. Ex. 6 | 40° C. 75% RH, open | 0.24% |
| | 60° C., closed | 0.44% |
| Comparative. Ex. 7 | 40° C. 75% RH, open | <0.05% |
| | 60° C., closed | 11.06% |

The results show that the amlodipine besylate in the granules obtained by wet processing amlodipine besylate with methyl cellulose was more stable than in the granules obtained by wet processing with other additives.

(Test 4)

Stability tests were conducted, in the same manner as in Test 1, on the granules obtained in Examples 12 and 13 and Comparative Example 8. The crystal form of the granules obtained in Examples 12 through 14 and Comparative Example 8 was also determined. The results are given in Tables 13 and 14.

TABLE 13

| | | Amount of |
|---|---|---|
| | | Degradation product |
| | Storage conditions | after 1 month |
| Comparative. Ex. 8 | 40° C. 75% RH, open | <0.05% |
| (control) | 60° C., closed | 1.02% |
| Example 12-1 | 40° C. 75% RH, open | <0.05% |
| | 60° C., closed | <0.05% |
| Example 12-2 | 40° C. 75% RH, open | <0.05% |
| | 60° C., closed | <0.05% |
| Example 13-1 | 40° C. 75% RH, open | <0.05% |
| | 60° C., closed | <0.05% |
| Example 13-2 | 40° C. 75% RH, open | <0.05% |
| | 60° C., closed | <0.05% |
| Example 13-2 | 40° C. 75% RH, open | <0.05% |
| | 60° C., closed | <0.05% |

TABLE 14

| | Crystal form |
|---|---|
| Comparative. Ex. 8 (control) | hydrate crystals |
| Example 12-1 | anhydrous crystals |
| Example 12-2 | anhydrous crystals |
| Example 13-1 | anhydrous crystals |
| Example 13-2 | anhydrous crystals |
| Example 13-3 | anhydrous crystals |
| Example 14-1 | anhydrous crystals |
| Example 14-2 | anhydrous crystals |

The above results show that anhydrous crystals resulted in no decomposition products. It is assumed that products kneaded with hydroxypropylmethyl cellulose will be as stable as those obtained with methyl cellulose, provided that the crystal form is anhydrous.

### INDUSTRIAL APPLICABILITY

The present invention can be used in the manufacture of pharmaceutical compositions that contain dihydropyridine compounds.

1.-17. (canceled)

18. A method for stabilizing a wet processed dihydropyridine compound, comprising wet processing the dihydropyridine compound with the use of a methylated cellulose-based polymer.

19. The method according to claim 18, wherein the dihydropyridine compound is amlodipine.

20. The method according to claim 18, wherein the dihydropyridine compound is amlodipine besylate.

21. The method according to any one of claims 18 to 20, wherein the methylated cellulose-based polymer is either methyl cellulose or hydroxypropylmethyl cellulose.

22. A method for preventing formation of hydrate crystals from a wet processed dihydropyridine compound, comprising wet processing the dihydropyridine compound with the use of a methylated cellulose-based polymer.

23. The method according to claim 22, wherein the dihydropyridine compound is amlodipine.

24. The method according to claim 22, wherein the dihydropyridine compound is amlodipine besylate.

25. The method according to any of claims 18, wherein the wet processing is either wet kneading or wet granulation.

26. A pharmaceutical composition with improved dihydropyridine compound stability, obtained by wet processing a dihydropyridine compound with the use of a methylated cellulose-based polymer.

27. The pharmaceutical composition according to claim 26, wherein the dihydropyridine compound is amlodipine.

28. The pharmaceutical composition according to claim 26, wherein the dihydropyridine compound is amlodipine besylate.

29. The composition according to any one of claims 26 to 28, wherein the methylated cellulose-based polymer is either methyl cellulose or hydroxypropylmethyl cellulose.

30. The pharmaceutical composition according to claim 26, wherein the wet processing is either wet kneading or wet granulation.

31. A granulated material with improved amlodipine besylate stability, obtained by wet granulating a mixture comprising amlodipine besylate and a methylated cellulose-based polymer.

32. A kneaded or granulated material with improved amlodipine besylate stability, obtained by adding an aqueous solution comprising water containing methyl cellulose to amlodipine besylate or a mixture comprising amlodipine besylate.
